(19) United States
(12) Patent Application Publication
(10) Pub. No.: US 2013/0303917 A1
(43) Pub. Date: Nov. 14, 2013

(54) ULTRASOUND PROBE AND ULTRASOUND DIAGNOSIS APPARATUS

(71) Applicants: Yasuhiro Ona, Nasushiobara-shi (JP); Hiroyuki Shikata, Nasushiobara-shi (JP); Takashi Takeuchi, Otawara-shi (JP); Takashi Kubota, Otawara-shi (JP); Hideki Kosaku, Nasushiobara-shi (JP); Yutaka Oonuki, Otawara-shi (JP)

(72) Inventors: Yasuhiro Ona, Nasushiobara-shi (JP); Hiroyuki Shikata, Nasushiobara-shi (JP); Takashi Takeuchi, Otawara-shi (JP); Takashi Kubota, Otawara-shi (JP); Hideki Kosaku, Nasushiobara-shi (JP); Yutaka Oonuki, Otawara-shi (JP)

(73) Assignees: Toshiba Medical Systems Corporation, Otawara-shi (JP); Kabushiki Kaisha Toshiba, Minato-ku, Tokyo (JP)

(21) Appl. No.: 13/979,865

(22) PCT Filed: Aug. 22, 2012
(86) PCT No.: PCT/JP2012/071163

§ 371 Date: Jul. 16, 2013

(30) Foreign Application Priority Data
Aug. 25, 2011 (JP) 2011-183216

(51) Int. Cl. A61B 8/00

(52) U.S. Cl. CPC A61B 8/4488; A61B 8/4444; A61B 8/4494; A61B 8/54
USPC 600/459

(57) ABSTRACT

An ultrasound probe and an ultrasound diagnosis apparatus are provided that are capable of reducing the frequency with which false images are generated in an ultrasound diagnostic image. An ultrasound probe of an embodiment comprises a transducer element unit in which multiple groups of transducer elements are arranged, and a selection means. Each of the multiple groups of transducer elements has a first to n^{th} subgroups of transducer elements. Emitting surfaces of the transducer elements included in the first to the n^{th} subgroups of transducer elements are arranged such that they face different directions for each subgroups of transducer elements. The selection means is provided to selectively activate a specific subgroup of transducer elements from among the first to the n^{th} subgroups of transducer elements, based on an external activation signal.

### TECHNICAL FIELD

Embodiments of the present invention relate to an ultrasound probe and an ultrasound diagnosis apparatus.

### BACKGROUND TECHNOLOGY

The ultrasound diagnosis apparatus uses an ultrasound probe to transmit ultrasonic waves into a subject and receive their reflected waves, thereby acquiring biological information about the subject.

The transmission/receiving of ultrasonic waves is carried out by multiple transducer elements provided on the ultrasound probe. Each transducer element is structured to have a pre-determined directivity. As a result, a structure ensuring broad directivity is required in order to generate ultrasonic waves across a wide area.

### PRIOR ART DOCUMENTS

### Patent Documents

[Patent Document 1] Japanese published unexamined application H10-146337

### SUMMARY OF THE INVENTION

### Problem to be Solved by the Invention

However, in ensuring directivity across a wide area, there may be some cases in which an area of high acoustic pressure (hereinafter referred to, in some cases, as a “grating lobe”) is generated in a direction different from that in which the generation of ultrasound is desired. Due to this grating lobe, a problem occurs in which a false image is generated in an ultrasound diagnostic image.

Embodiments are designed to solve the problem described above, with the aim of providing an ultrasound probe and ultrasound diagnosis apparatus that are capable of reducing the frequency with which false images are generated in an ultrasound diagnostic image.

### Means of Solving the Problem

An ultrasound probe of an embodiment comprises a transducer element unit in which multiple groups of transducer elements are arranged, and a selection means. Each of the multiple groups of transducer elements has a first to n^{th} subgroups of transducer elements. Emitting surfaces of the transducer elements included in the first to the n^{th} subgroups of transducer elements are arranged such that they face different directions for each subgroups of transducer elements. The selection means is provided to selectively activate a specific subgroup of transducer elements from among the first to the n^{th} subgroups of transducer elements, based on an external activation signal.

In addition, an ultrasound diagnosis apparatus of an embodiment comprises an ultrasound probe, a transmission/reception means, and a selection control means. The ultrasound probe comprises a transducer element unit in which multiple groups of transducer elements are arranged, along with a selection means. Each of the multiple groups of transducer elements comprises a first to n^{th} subgroups of transducer elements. Emitting surfaces of the transducer elements included in the first to the nth subgroups of transducer elements are arranged such that they face different directions for each subgroup of transducer elements. The selection means is provided to selectively activate a specific subgroup of transducer elements from among the first to the n^{th} subgroups of transducer elements, based on an activation signal. The transmission/reception means causes the transmission/reception of ultrasonic waves by sending the activation signal to the transducer elements included in the first to the n^{th} subgroups of transducer elements. The selection control means controls the operation of the selection means.

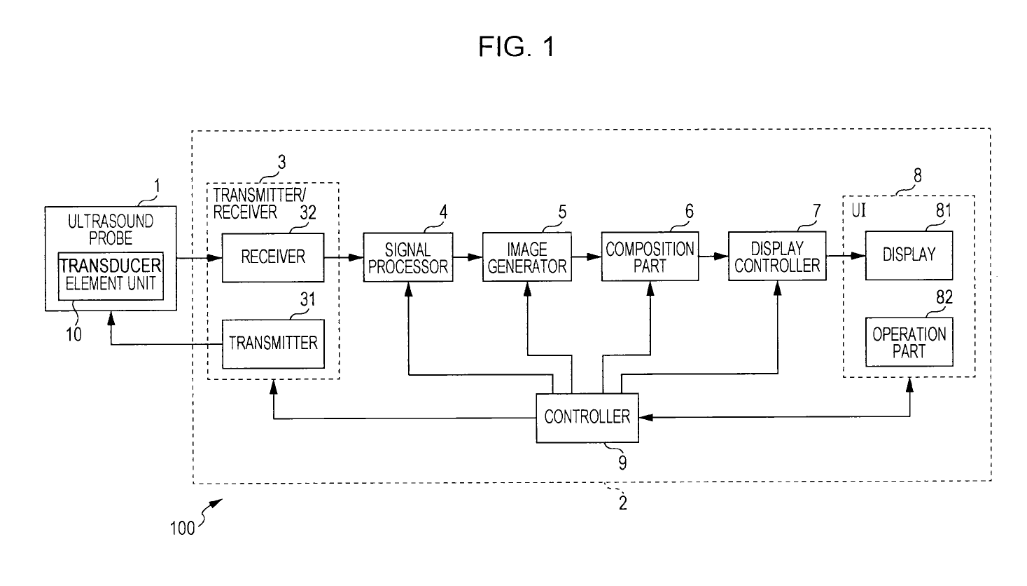

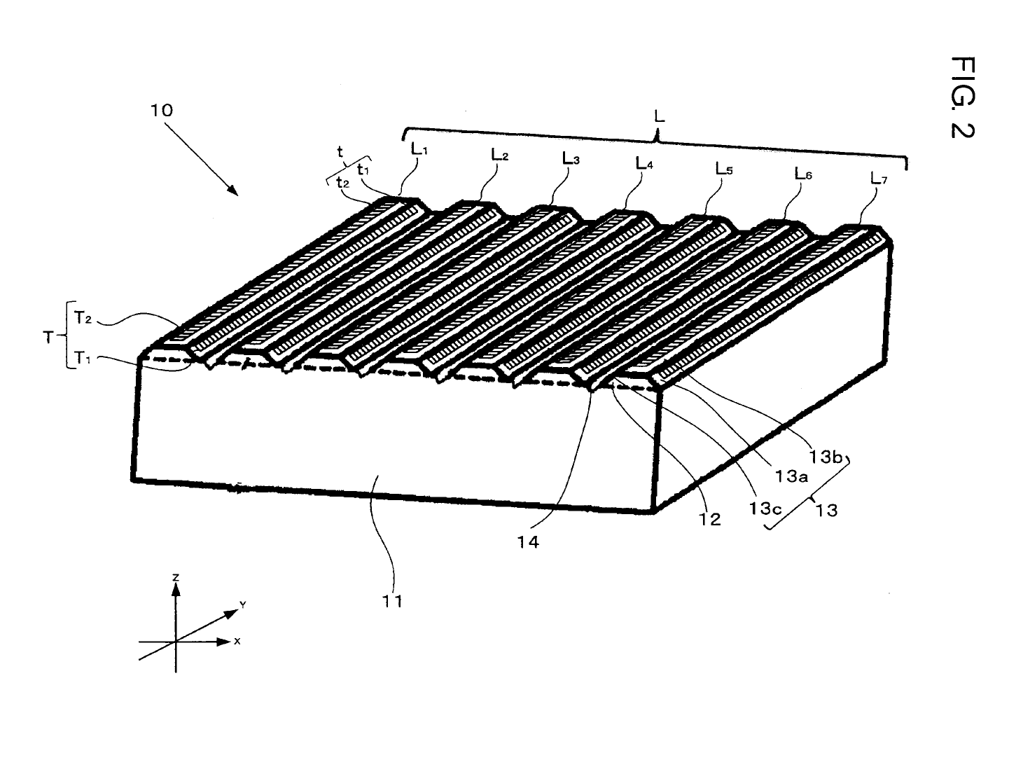

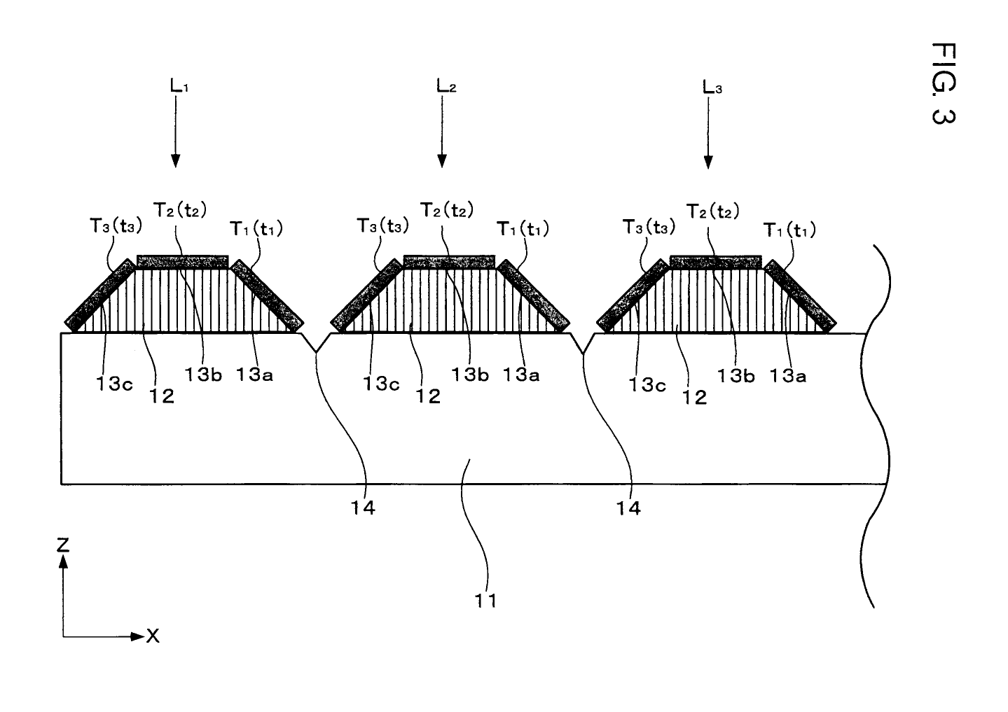

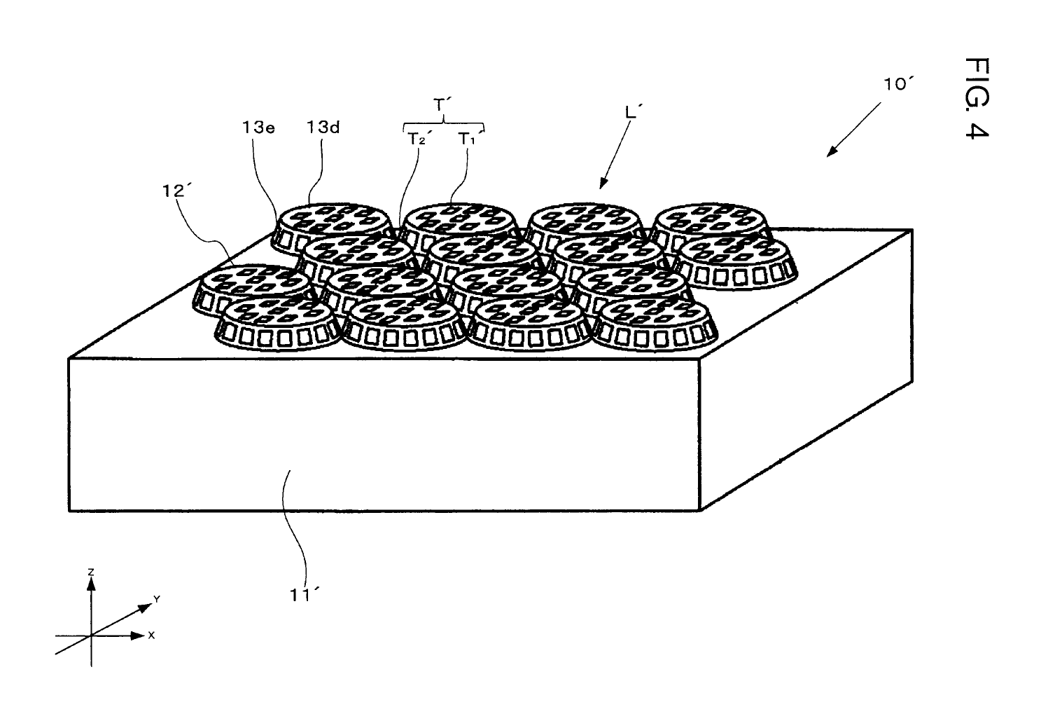

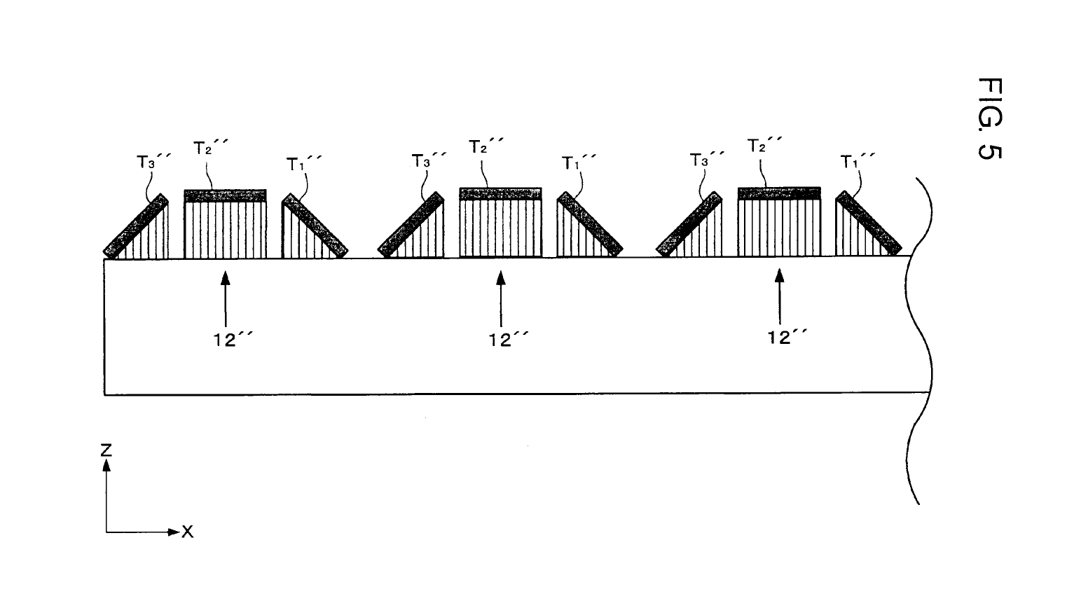

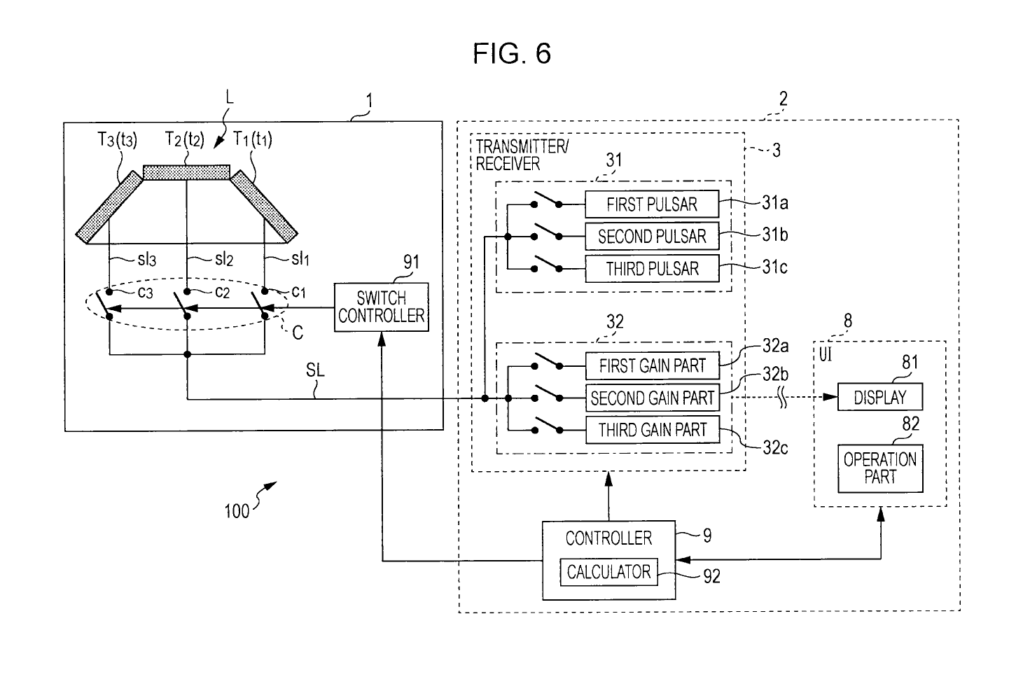

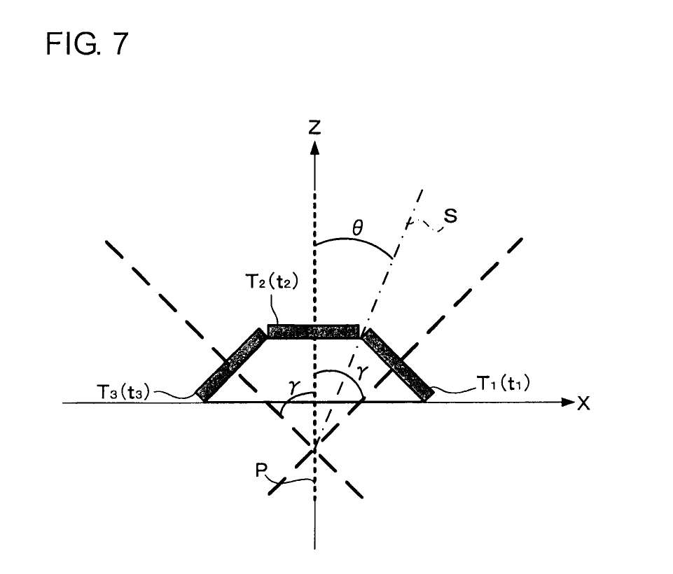

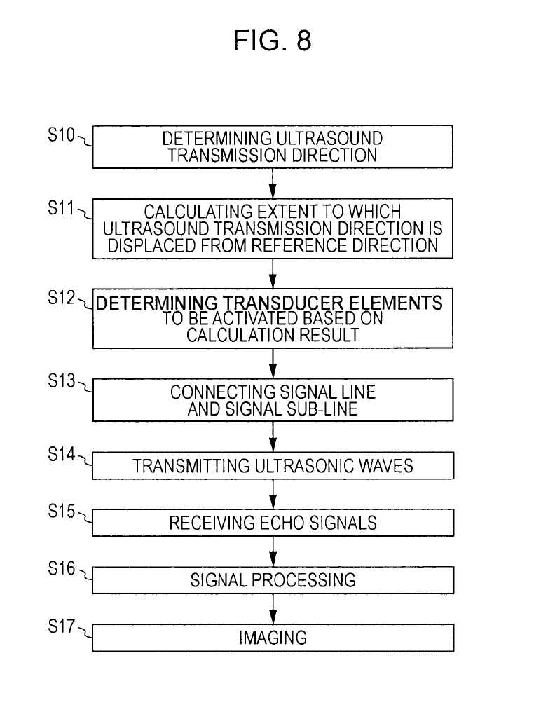

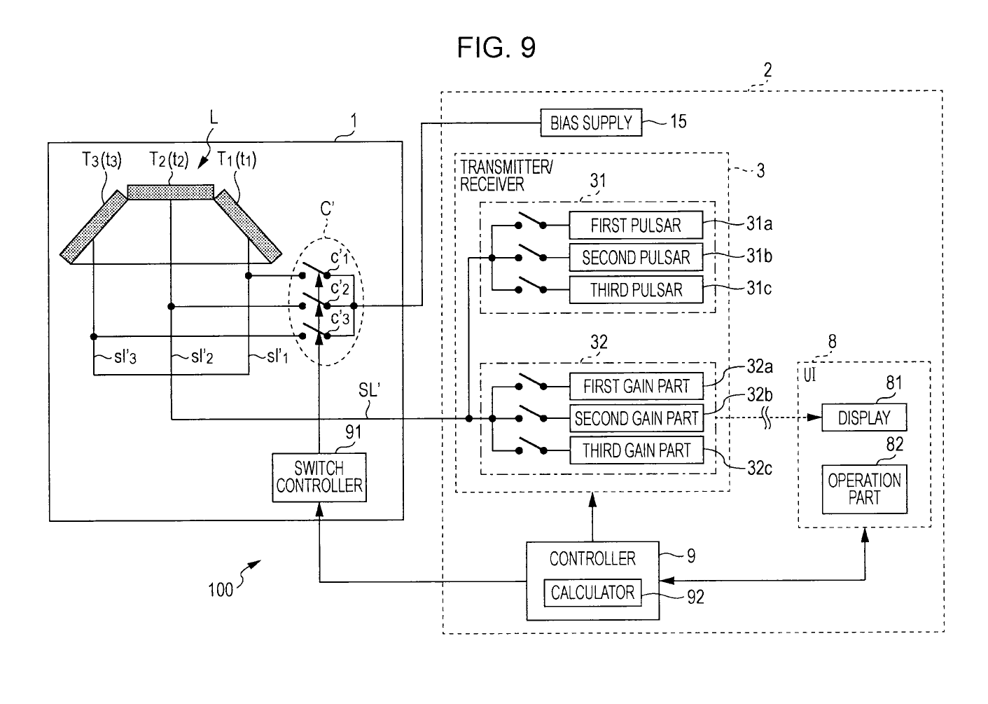

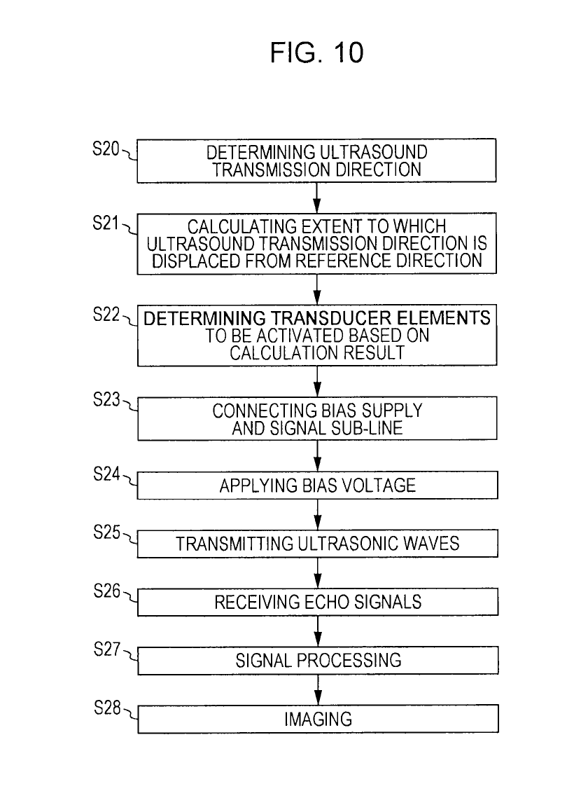

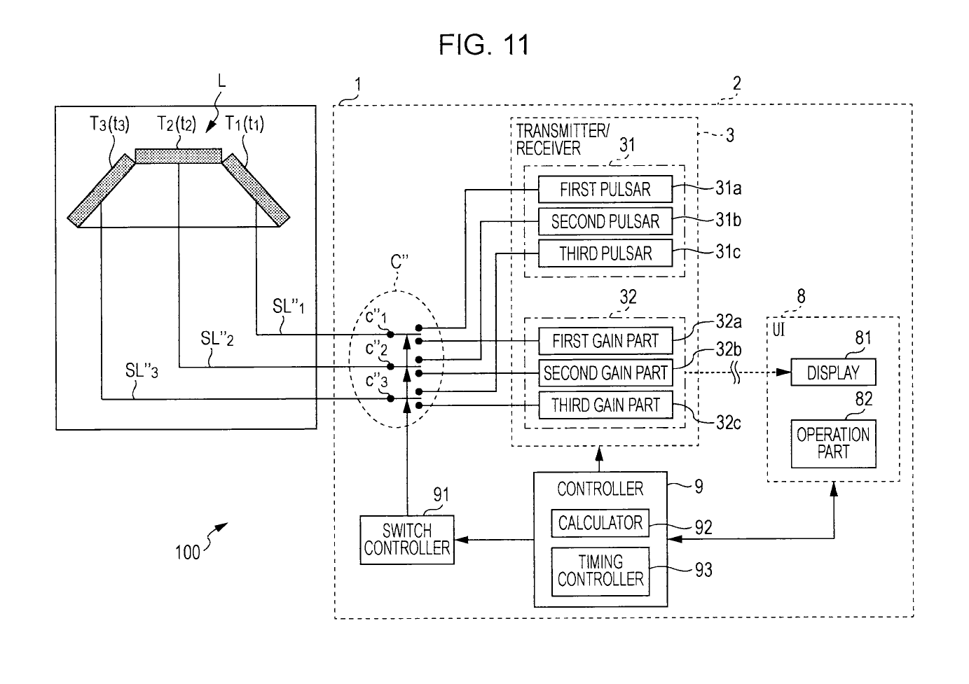

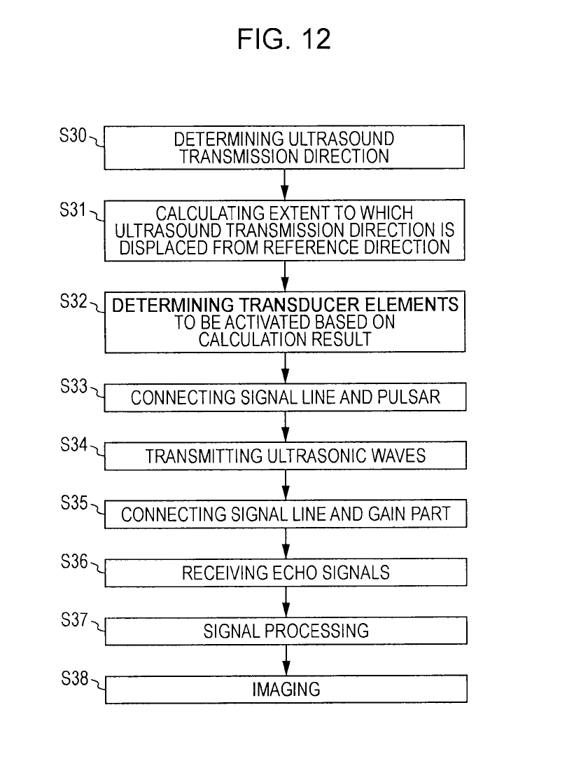

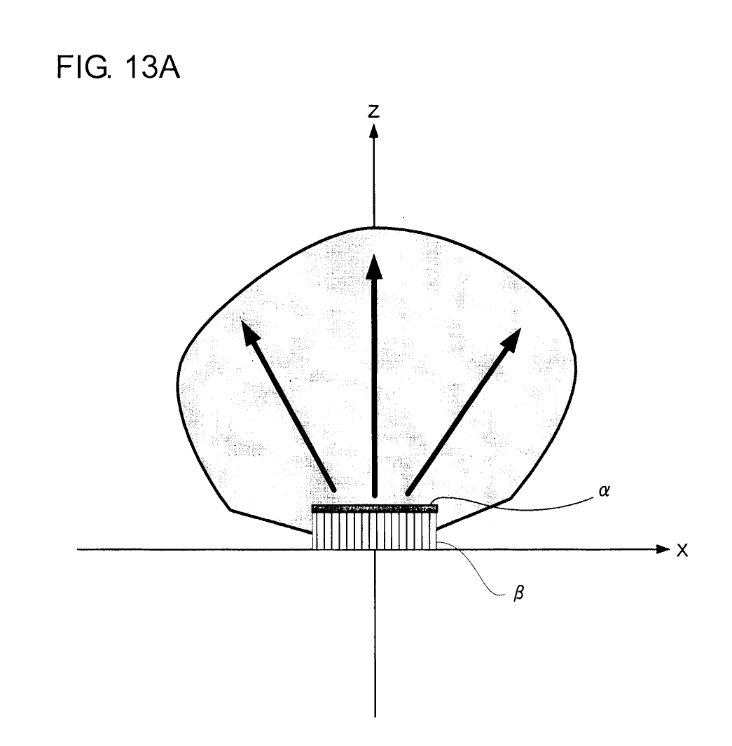

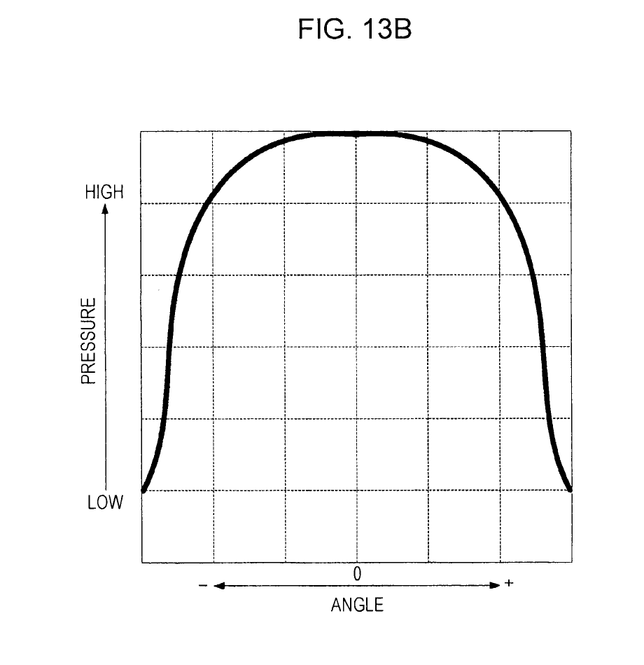

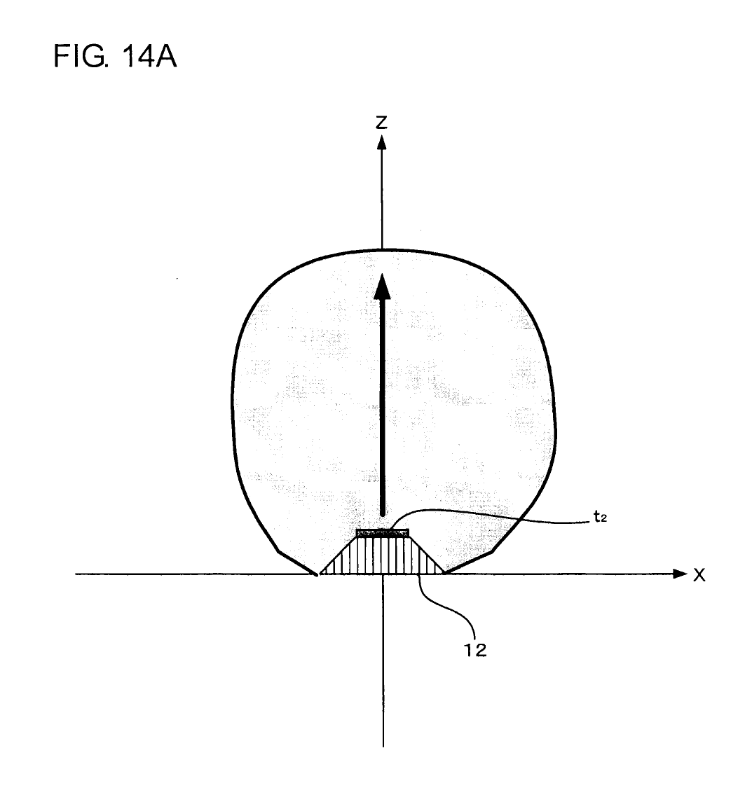

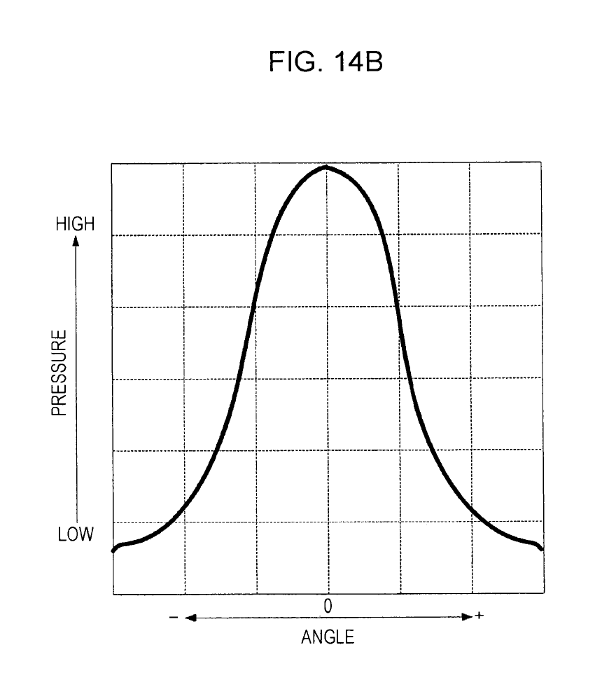

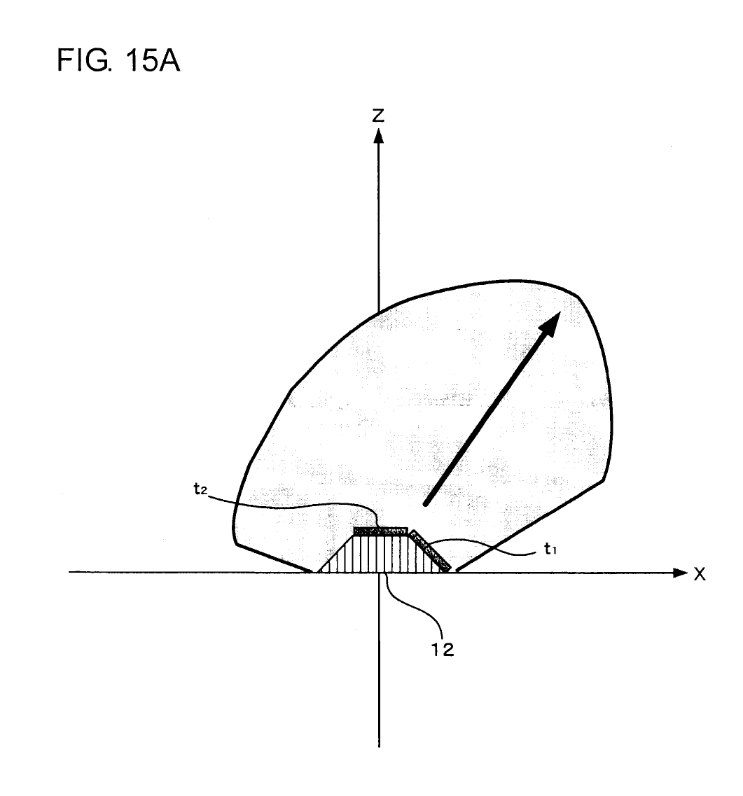

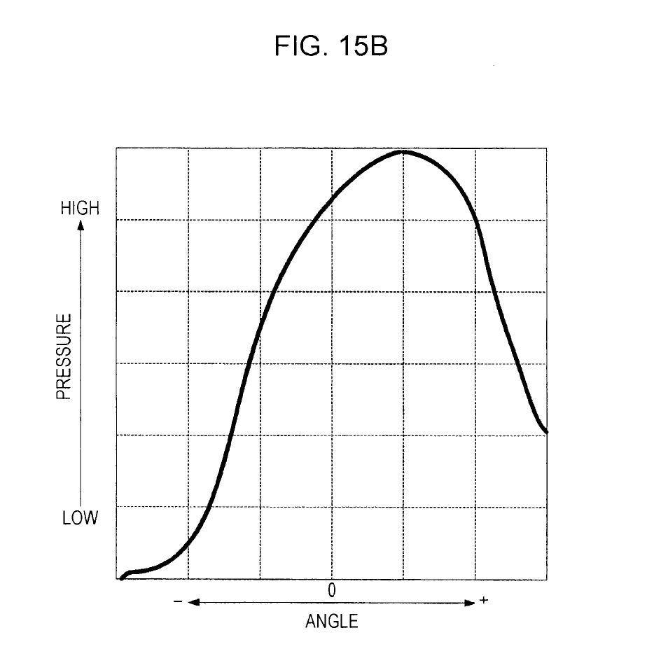

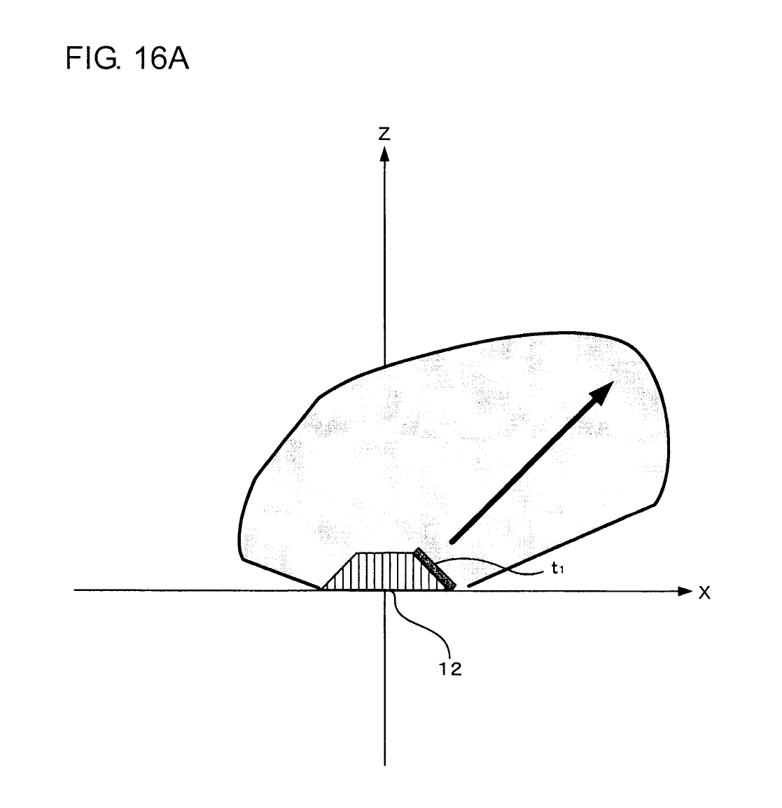

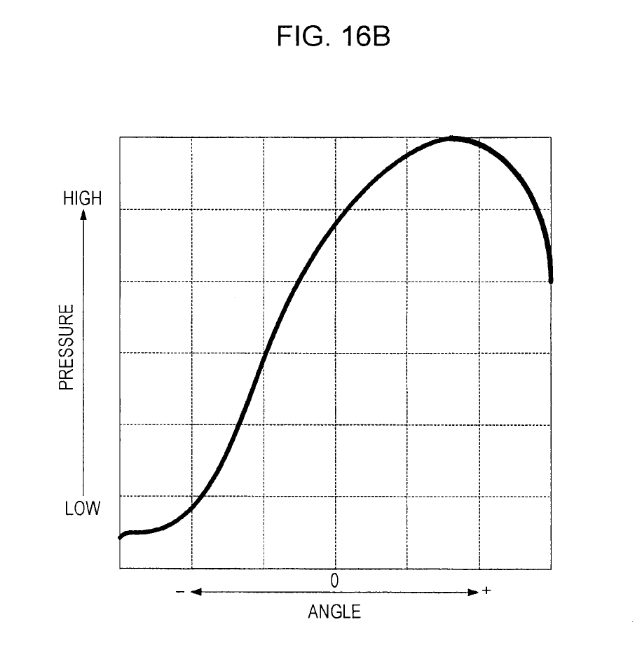

### BRIEF DESCRIPTION OF THE DRAWINGS

[FIG. 1] is a block diagram illustrating an outline of the ultrasound diagnosis apparatus common to the embodiments.

[FIG. 2] is a diagram illustrating one example of the configuration of a transducer element unit common to the embodiments.

[FIG. 3] is a diagram illustrating one example of the configuration of the transducer element unit common to the embodiments.

[FIG. 4] is a diagram illustrating another example of the configuration of the transducer element unit common to the embodiments.

[FIG. 5] is a diagram illustrating a further example of the configuration of the transducer element unit common to the embodiments.

[FIG. 6] is a block diagram illustrating the detailed configuration of the ultrasound diagnosis apparatus in the first embodiment.

[FIG. 7] is a diagram providing a supplemental description of the ultrasound diagnosis apparatus in the first embodiment.

[FIG. 8] is a flow chart illustrating the operation of the ultrasound diagnosis apparatus in the first embodiment.

[FIG. 9] is a block diagram illustrating the detailed configuration of the ultrasound diagnosis apparatus in the second embodiment.

[FIG. 10] is a flow chart illustrating the operation of the ultrasound diagnosis apparatus in the second embodiment.

[FIG. 11] is a block diagram illustrating the detailed configuration of the ultrasound diagnosis apparatus in the third embodiment.

[FIG. 12] is a flow chart illustrating the operation of the ultrasound diagnosis apparatus in the third embodiment.

[FIG. 13A] is a diagram describing the configuration of a comparative example.

[FIG. 13B] is a graph describing the comparative example.

[FIG. 14A] is a diagram describing the configuration of an example.

[FIG. 14B] is a graph describing the example.

[FIG. 15A] is a diagram describing the configuration of an example.

[FIG. 15B] is a graph describing the example.

[FIG. 16A] is a diagram describing the configuration of an example.

[FIG. 16B] is a graph describing the example.

### MODE FOR CARRYING OUT THE INVENTION

A configuration of an ultrasound diagnosis apparatus common to the first to third embodiments is described with reference to FIGS. 1 through 4.

### <Composition of the Ultrasound Diagnosis Apparatus>

FIG. 1 is a block diagram of an ultrasound diagnosis apparatus 100. The ultrasound diagnosis apparatus 100 comprises an ultrasound probe 1 and a main unit 2. The main unit 2 comprises a transmitter/receiver 3, a signal processor 4, an image generator 5, composition part 6, a display controller 7, a user interface (UI) 8, and a controller 9. The ultrasound probe 1 and the main unit 2 are connected by a cable attached to the ultrasound probe 1 (not illustrated).

### (Ultrasound Probe 1)

The ultrasound probe 1 has a transducer element unit 10 in which multiple groups of transducer elements are arranged, with each group comprising a predetermined number of transducer elements. The ultrasound probe 1 transmits ultrasonic waves to a subject via being connected to the main unit 2, and can receive waves reflected from the subject as echo signals. The detailed configuration of the transducer element unit 10 is provided below.

### (Transmitter/Receiver 3)

The transmitter/receiver 3 provides an activation signal to the ultrasound probe 1, causing it to generate ultrasonic waves, while receiving the echo signals received by the ultrasound probe 1. The transmitter/receiver 3 outputs the received echo signals to the signal processor 4. The transmitter/receiver 3 comprises a transmitter 31 and a receiver 32. In the embodiment, multiple transmitter/receivers 3 are provided, corresponding to the number of transducer elements. The transmitter/receiver 3 may also be provided within the ultrasound probe 1. In the embodiment, the transmitter/receiver 3 is one example of the “transmission/reception means.”

### (Transmitter 31)

The transmitter 31 provides an activation signal to the ultrasound probe 1, causing it to generate ultrasonic waves. The transmitter 31 provides an activation signal to the ultrasound probe 1, causing it to transmit beamformed ultrasonic waves to the specified focal point. The beamform corresponding to the specified focal point may, for example, be carried out by phase matching between an acoustic lens (not illustrated) and the array direction (the X direction as described later). The transmitter 31 may include, for example, a clock generator (not illustrated), a transmission delay circuit, and a pulsar circuit. The clock generator generates clock signals that determine the transmission timing and the transmission frequency of the ultrasound signals. The transmission delay circuit performs the transmission focusing by delaying at the time of the ultrasound transmission in response to the convergence delay time for converging the ultrasonic waves at a specified depth, as well as to the deflection delay time for transmitting the ultrasonic waves in the specified direction. In the embodiment, particularly, control of the deflection delay time for transmitting the ultrasonic waves in the specified direction, is important. The pulsar circuit comprises a number of pulsars equivalent to the number of individual channels corresponding to the ultrasound transducer elements. The pulsar circuit generates an activation pulse (activation signal) at the transmission timing to which the delay is applied, supplying an activation pulse (activation signal) to the transducer elements in ultrasound probe 1.

### (Receiver 32)

The receiver 32 receives echo signals received by the ultrasound probe 1. The receiver 32 performs a delay processing to the echo signals, thereby converting the analog echo signals into digital data which is phased and added. The receiver 32 comprises, for example, a gain circuit, an A/D converter, a reception delay circuit and an adder (not illustrated). The gain circuit amplifies (applies gain) in each receiving channel to the echo signals output via the transducer elements of the ultrasound probe 1. The A/D converter converts the amplified echo signals to digital signals. The reception delay circuit provides the necessary delay time in order to determine the receiving directivity for the digitally converted echo signals. Specifically, the reception delay circuit provides the digital echo signals with a convergence delay time for converging the ultrasonic waves from the specified depth, and with a deflection delay time for setting the receiving directivity in the specified direction. This delay time is not necessarily a simple calculation of the amount of convergence delay and the amount of deflection angle delay, but is calculated based on specified formulas and principles. The adder adds the echo signals, to which a delay has been applied. This addition allows emphasis of the reflection component from the direction corresponding to receiving directivity. In other words, the echo signals obtained from the specified direction are phased and added by the reception delay circuit and the adder. The receiver 32 outputs the echo signals, to which a delay processing is applied, to the signal processor 4.

### (Signal Processor 4)

The signal processor 4 performs various types of signal processing corresponding to the echo signals output from the transmitter/receiver 3. For example, the signal processor 4 comprises a B-mode processor. The B-mode processor receives the echo signals from the transmitter/receiver 3 and processes vibration amplitude information from the echo signals into visual images. Specifically, the B-mode processor performs band-pass filter processing on the echo signals; subsequently, it detects the envelope curve of the output signal and performs logarithmic conversion on the detected data in order to compress it. Furthermore, the signal processor 4 may also comprise a CFM (Color Flow Mapping) processor. The CFM processor produces visual images of blood flow information. Blood flow information may include speed, distribution or power. Furthermore, the signal processor 4 may comprise a Doppler processor. The Doppler processor extracts Doppler shift frequency components by performing phase detection of the echo signals, and performs FFT processing to generate a Doppler frequency distribution that illustrates the speeds of blood flows. The signal processor 4 outputs the signal-processed echo signals (ultrasound rasterized data) to the image generator 5.

### (Image Generator 5)

The image generator 5 generates ultrasound image data based on the signal-processed echo signals (ultrasound rasterized data) that is output from the signal processor 4. The image generator 5 may comprise, for example, a DSC (Digital Scan Converter). The image generator 5 converts the signal-processed echo signals represented by signal sequences of scanning lines into image data represented by an orthogonal coordinate system (scan conversion processing). The image generator 5 performs the scan conversion processing on the echo signals that have been signal processed by the B-mode processor, resulting in the generation of B-mode image data that represents the form of a tissue of the subject. The image generator 5 outputs the ultrasound image data to the composition part 6.

For example, the ultrasound probe 1 and the transmitter/receiver 3 scan a cross-section of the inside of the subject using ultrasonic waves, and the image generator 5 generates B-mode image data (tomographic imaging data), representing the two-dimensional shape of a tissue in the cross-section. Furthermore, the ultrasound probe 1 and the transmitter/receiver 3 may scan a three-dimensional area using ultrasonic waves to obtain volume data. In this case, the image generator 5 may perform volume rendering of the volume data, with the aim of generating 3D image data representing three-dimensional shape of a tissue. Further, the image generator 5 may perform MPR (Multi Planar Reconstruction) processing on the volume data, generating image data of an arbitrary cross-section (MPR image data).

The ultrasound diagnosis apparatus described in the embodiment may be provided with image storage (not illustrated). The image storage stores data obtained from the ultrasound diagnosis apparatus described in the embodiment. For example, the image storage may store echo signals output from the transmitter/receiver 3. Additionally, the image storage may store ultrasound rasterized data output from the signal processor 4. Furthermore, the image storage may store ultrasound image data, such as tomographic imaging data, output from the image generator 5.

### (Composition Part 6)

The composition part 6 composes multiple ultrasound image data to generate composite image data. The composition of image data by the composition part 6 is carried out using commonly known methods. For example, the composite image data may be generated by weighting to each of the multiple ultrasound image data in accordance with the depth at which the ultrasound image data has been obtained, and adding and averaging these images. The composition part 6 outputs the composite image data to the display controller 7.

### (Display Controller 7)

The display controller 7 receives composite image data from the composition part 6 and displays composite images based on the composite image data on the display 81.

### (User Interface 8)

The user interface (UI) 8 includes a display 81 and an operation part 82. The display 81 comprises a monitor, such as a CRT or a liquid crystal display. The operation part 82 comprises an input equipment, such as a keyboard or a mouse.

### (Controller 9)

The controller 9 controls operations of various parts of the ultrasound diagnosis apparatus 100. The controller 9 controls, for example, the transmission/receiving of ultrasonic waves by sending delay signals to the transmitter/receiver 3.

It should be noted that the functions of each of the image generator 5, the composition part 6 and the display controller 7 may be performed by programs. As an example of this, each of the image generator 5, composition part 6, and display controller 7 may be composed from CPU, GPU, ASIC or other processing equipment (not illustrated) along with ROM, RAM, HDD or other storage equipment (not illustrated). The storage equipment stores an image generation program used to perform the functions of the image generator 5, a composition program used to perform the functions of the composition part 6, and a display processing program used to perform the functions of the display controller 7. The processing equipment such as a CPU, etc. performs the functions of each part by performing each program stored in the storage equipment.

### <Configuration of Transducer Element Unit>

The following is a description of the configuration of the transducer element unit 10, which is common to the embodiments, with reference to FIGS. 2 and 3. FIG. 2 is a perspective view of the transducer element unit 10. FIG. 3 is an enlarged diagram of an X-Z cross-section of the transducer element 10. It should be noted that, in FIG. 3, a part of the transducer element unit 10 is abbreviated from the diagram.

As illustrated in FIG. 2, the transducer element unit 10 is structured to include a seat 11 and multiple groups of transducer elements L.

The seat 11 is formed from a quartz (SiO₂) substrate, a silicon (Si) substrate, or other materials suitable for use in semiconductor processing. The multiple groups of transducer elements L are arranged on the upper surface of the seat 11. In the embodiment, seven groups of transducer elements L₁ to L₇, each of which is formed in a seriate shape, are arranged in arrays on the base, but the number of groups of transducer elements L is not limited to this. In the embodiment, the scanning direction of the transducer element unit 10 is defined as X direction, while the direction perpendicular to the scanning direction along the upper surface of the seat 11 (the “elevation direction”) is defined as Y direction, and the depthwise direction of the seat 11 is defined as Z direction.

As illustrated in FIGS. 2 and 3, each of the multiple groups of transducer elements L comprises a base 12 and subgroups of transducer elements T. In the embodiment, each of the groups of transducer elements L has three subgroups of transducer elements T₁ to T₃. The number of subgroups of transducer elements T is not restricted to three (in general, it is possible to provide the first to the n^{th} subgroup of transducer elements (n≧2)).

The bases 12 are arranged in rows on the upper surface of the seat 11, and include a surface 13 on which the subgroups of transducer elements T are arranged. In the embodiment, the base 12 is formed in a convex shape. In the embodiment, additionally, three surfaces 13 (surfaces 13a, 13b and 13c) are formed, in accordance with the number of subgroups of transducer elements T. A subgroup of transducer elements T₁ is positioned on the surface 13a, a subgroup of transducer elements T₂ is positioned on the surface 13b, and a subgroup of transducer elements T₃ is positioned on the surface 13c.

The subgroups of transducer elements T each comprise multiple transducer elements t. In the embodiment, the multiple transducer elements t are arranged in a single row in the elevation direction (Y direction). Furthermore, in the embodiment, the transducer elements included in the subgroup of transducer elements T₁ are defined as t₁, the transducer elements included in the subgroup of transducer elements T₂ are defined as t₂, and the transducer elements included in the subgroup of transducer elements T₃ are defined as t₃.

The transducer elements t receive signals from the transmitter and transmit ultrasonic waves from their emitting surfaces. Additionally, the transducer elements t receive echo signals from the subject, and send them to the receiver 32. In the state that the multiple subgroups of transducer elements T are arranged on the base 12, the emitting surfaces of the transducer elements t in each subgroup of transducer elements T are arranged to face a different direction for each of the subgroups of transducer elements T. In other words, in the embodiment, the emitting surfaces of the transducer elements t₁, t₂ and t₃ are arranged to face different directions. It should be noted that, in the embodiment, the emitting surfaces of each transducer elements t in the multiple groups of transducer elements L are arranged to face the same direction (for example, the emitting surface of the transducer element t₁ in group of transducer elements L₁ and the emitting surface of the transducer element t₁ in the group of transducer elements L₂ face the same direction.)

As the transducer element t, a piezoelectric material or an MUT (Micromachining Ultrasound Transducer) element may be used. An MUT element can include a cMUT (Capacitive Micromachining Ultrasound Transducer) and a pMUT (Piezoelectric Micromachining Ultrasound Transducer).

In the embodiment, grooves 14 are provided between the bases 12. By providing the grooves 14, it is possible to acoustically separate the subgroups of transducer elements T positioned on the respective bases 12.

### <Modified Example of a Transducer Element Unit>

The configuration of the transducer element unit 10 is not restricted to that illustrated in FIG. 2. FIG. 4 illustrates a perspective view of a transducer element unit 10′. FIG. 5 illustrates an enlarged diagram of an X-Z cross-section of a transducer element unit 10″.

As illustrated in FIG. 4, for example, the multiple groups of transducer elements L′ (16 groups in FIG. 4) may be arranged on cylindrical bases 12′ positioned in a matrix formation. In this case, the subgroups of transducer elements T′ (T₁′ and T₂′) are arranged on the upper surfaces 13d and the side surfaces 13e of the cylinders.

Alternatively, as illustrated in FIG. 5, the base 12″ may be divided into three parts, with the subgroups of transducer elements T₁″ to T₃″ arranged on each of the parts.

As described, it is sufficient if the base 12 is structured such that the multiple subgroups of transducer elements T can be positioned thereupon. More specifically, it is sufficient if the base 12 is structured such that the emitting surfaces of the transducer elements t are positioned thereupon in the way that they face different directions for each subgroup of transducer elements T. Consequently, the base 12 may be either convex or concave. Furthermore, the base 12 may be integrally composed with the seat 11.

### FIRST EMBODIMENT

The following is a description of the ultrasound diagnosis apparatus in the first embodiment, with reference to FIGS. 6 to 8. FIG. 6 is a block diagram illustrating an outline of the ultrasound diagnosis apparatus for the purpose of explaining the embodiment. FIG. 6 illustrates only one group of transducer elements L. It also omits part of the configuration. FIG. 8 is a flow chart indicating an operation of the ultrasound diagnosis apparatus.

### [Configuration of Ultrasound Probe]

In the embodiment, the ultrasound probe 1 comprises: multiple groups of transducer elements L (for example, 96 groups); the number of signal lines SL equivalent to the number of the multiple groups of transducer elements L; signal sub-lines sl corresponding to the subgroups of transducer elements T included in one group of transducer elements L; the number of connectors C equivalent to the number of the multiple groups of transducer elements L; and a switch controller 91. It should be noted that FIG. 6 illustrates one group of transducer elements L, one signal line SL, one connector C and signal sub-lines sl₁ to sl₃.

One end of the signal line SL is connected to the transmitter/receiver 3 (the transmitter 31 and the receiver 32) of the main unit 2, while the other end thereof can be connected via the connector C to at least one of the signal sub-lines sl₁ to sl₃. It should be noted that the signal line SL is connected via a cable (not illustrated) to the transmitter/receiver 3.

One ends of the signal sub-lines sl₁ to sl₃ can be connected to the other end of the signal line SL, while the other ends thereof are connected to the subgroups of transducer elements T₁ to T₃, respectively. It should be noted that, in general, the signal sub-lines sl include the first signal sub-line sl₁ to the n^{th} signal sub-line slₙ (where n≧2), in accordance with the number of the subgroups of transducer elements T.

The connector C is used to selectively activate a specified subgroup of transducer elements from among the multiple subgroups of transducer elements T, based on an activation signal from the outside such as the main unit 2, etc. Specifically, the connector C is configured to include a number of switches c equivalent to the number of the signal sub-lines sl. In the embodiment, three switches c₁ to c₃ are provided corresponding to the signal sub-lines sl₁ to sl₃, respectively. The switches c switch the connection and disconnection between the signal line SL and the signal sub-lines sl. It should be noted that it is sufficient if at least one connector C is provided with regard to the multiple groups of transducer elements L. The connector C in the embodiment is one example of the “selection means.” Furthermore, the switches c₁ to c₃ in the embodiment are an example of the “first switching means.”

The switch controller 91 controls the operation of the connector C. Specifically, the switch controller 91 performs the switching of each of the switches c. For example, when a delay signal is transmitted from the controller 9 in order to activate the subgroup of transducer elements T₁ alone, the switch controller 91 activates the switch c₁ based on this delay information, connecting the signal line SL and the signal sub-line sl₁. In this state, the subgroup of transducer elements T₁ receives the signal (the activation signal) transmitted from the transmitter 31 (a first pulsar 31a), and is able to transmit ultrasonic waves. It should be noted that there are no restrictions requiring the switch controller 91 to only operate one switch c. One switch controller 91 may simultaneously operate multiple switches c (for example, c₁ and c₂). As a result, it becomes possible to simultaneously activate the multiple subgroups of transducer elements (for example T₁ and T₂). The switch controller 91 in the embodiment is an example of the “selection control means.”

It is sufficient if at least one switch controller 91 is provided with regard to the multiple groups of transducer elements L. If the switch controller 91 is provided for each of the multiple groups of transducer elements L, differing controls can be performed for each group of transducer elements L. For example, it is possible to operate the switch c₁ to activate only the subgroup of transducer elements T₁ within the group of transducer elements L₁, and to operate the switch c₃ to activate only the subgroup of transducer elements T₃ within the group of transducer elements L₂.

### <Configuration of Transmitter>

In the embodiment, the transmitter 31 comprises, as a pulsar, a first pulsar 31a, a second pulsar 31b and a third pulsar 31c. The pulsars generate different pulses.

One pulsar is selected from among the first pulsar 31a, the second pulsar 31b and the third pulsar 31c based on the delay signal from the controller 9. The selected pulsar generates an activation signal, and supplies the activation signal via the signal line SL and the signal sub-lines sl to the transducer elements t in the subgroups of transducer elements T.

### <Configuration of Receiver>

In the embodiment, the receiver 32 comprises, as a gain circuit, a first gain part 32a, a second gain part 32b and a third gain part 32c. The gain parts apply different gains to the echo signals.

One gain part is selected from among the first gain part 32a, the second gain part 32b and the third gain part 32c based on the delay signal from the controller 9. The selected gain part applies the specified gain to the echo signals received by the transducer elements t, and transmits them to the signal processor 4 or other post-processing part.

### <Configuration of Controller>

In the embodiment, the controller 9 is configured to include a calculator 92.

The calculator 92 calculates the extent to which the direction of ultrasound transmission, input by the operation part 82 etc., is displaced from the reference direction. The reference direction is the arbitrarily set ultrasound transmission direction for the groups of transducer elements L.

The controller 9 determines a subgroup of transducer elements to be activated based on the calculation results of the calculator 92, and based on this, transmits delay information (information for switching control) to the switch controller 91. The processes carried out by the calculator 92 and the controller 9 may be performed for each group of transducer elements L. In other words, the controller 9 may transmit differing delay signals with regard to each of the groups of transducer elements L.

The following is a specific example of the processing by the calculator 92 and the controller 9, using FIG. 7. FIG. 7 is an X-Z cross-sectional view of one group of transducer elements L, seen from the side (Y direction). Here, the direction in which ultrasonic waves are transmitted is denoted by “S” while the reference direction is denoted by “P”. Moreover, the angle corresponding to the reference direction is denoted by “θ”. Furthermore, the angle between the ultrasound beams transmitted from the transducer elements t within one group of transducer elements L is denoted by “γ”.

Firstly, the calculator 92 calculates the angle θ of the ultrasound transmission direction S with regard to the reference direction P. The controller 9 selects the transducer elements t (subgroup of transducer elements T) to be activated based on the relationship between the angle θ and the angle γ. The relationship between the angle θ and the angle γ is predetermined as illustrated, for example, in the table in Table 1.

TABLE 1

| | Transducer elements to be |
|---|---|
| | activated |
| θ > γ | Transducer elements t₁ |
| γ > θ > −γ | Transducer elements t₂ |
| −γ > θ | Transducer elements t₃ |

### <Operation>

Next, the following is a description of the operation of the ultrasound diagnosis apparatus 100 in the embodiment, with reference to FIG. 8. It should be noted that the reference direction P is predetermined.

The controller 9 determines the ultrasound transmission direction S based on the scan mode of ultrasound beams (S 10).

The calculator 92 calculates the extent (angle θ) to which the ultrasound transmission direction S determined in S10 is displaced from the reference direction P (S11).

The controller 9 determines the transducer elements t to be activated based on the calculation results in S11, from table data, etc. (S 12). Here, it is assumed that transducer elements t₁ are selected.

The controller 9 transmits a delay signal and delay information based on the determination result in S12, to the switch controller 91 and the transmitter/receiver 3. The switch controller 91 activates the switch c₁ based on the delay information, connecting the signal line SL and the signal sub-line sl₁ (S13).

The transmitter 31 of the transmitter/receiver 3 activates the first pulsar 31a based on the delay signal, and transmits an activation signal to the transducer elements t₁. Based on the activation signal, the transducer elements t₁ transmit ultrasonic waves to the subject (S14).

The receiver 32 of the transmitter/receiver 3 activates the first gain part 32a based on the delay signal, then applies the specified gain to the echo signal and receives it (S 15).

The echo signal to which gain has been applied in S15 is transmitted to the signal processor 4, etc., (not illustrated in FIG. 6), and once it has been subjected to the specified process (S16), the composition part 6 acquires image data (S17). The display 81 displays an image based on the image data acquired in S17.

### <Operation and Effect>

The following is an explanation of the operation and effect of the embodiment.

The ultrasound probe 1 in the embodiment has a transducer element unit 10 in which multiple groups of transducer elements L (for example, L₁ to L₉₆) are arranged. Each of the multiple groups of transducer elements L has the first to the n^{th} subgroups of transducer elements T (T₁ to Tₙ). The emitting surfaces of the transducer elements t (t₁ to tₙ) included in the first to the n^{th} subgroups of transducer elements T (T₁ to Tₙ) are arranged such that they face different directions. Furthermore, the ultrasound probe 1 has a selection means (connector C). The selection means (connector C) is designed to selectively activate a specified subgroup of transducer elements T from among the first to the n^{th} subgroups of transducer elements T (T₁ to Tₙ), based on an external activation signal.

Furthermore, the ultrasound diagnosis apparatus 100 in the embodiment includes an ultrasound probe 1, a transmission/reception means (transmitter/receiver 3) and a selection control means (switch controller 91). The ultrasound probe 1 has a transducer element unit 10 in which multiple groups of transducer elements L (for example, L₁ to L₉₆) are arranged. Each of the multiple groups of transducer elements L has the first to the n^{th} subgroups of transducer elements T (T₁ to Tₙ). The emitting surfaces of the transducer elements t (t₁ to tₙ) in the first to the n^{th} subgroups of transducer elements T (T₁ to Tₙ) are arranged such that they faces different directions for each subgroups of transducer elements T. Furthermore, the ultrasound probe 1 has a selection means (connector C). The selection means (connector C) is designed to selectively activate a specified subgroup of transducer elements T from among the first to the n^{th} subgroups of transducer elements T (T₁ to Tₙ), based on an activation signal. The transmission/reception means (transmitter/receiver 3) transmits the activation signal to the transducer elements t (t₁ to tₙ) included in the first to the n^{th} subgroups of transducer elements T (T₁ to Tₙ), in order to perform the transmission/receiving of ultrasonic waves. The selection control means (switch controller 91) controls the operation of the selection means (controller C).

Configuring the equipment in this manner allows only the transducer elements that are positioned in the direction in which the operator wishes to transmit/receive ultrasound for each of the multiple groups of transducer elements L to be activated, resulting in that it is difficult to form a grating lobe. As a result, it is possible to reduce the frequency with which false images occur in an ultrasound diagnostic image.

Furthermore, the ultrasound probe 1 in the embodiment has signal lines SL and the first to the n^{th} signal sub-lines sl. The number of signal lines SL is equivalent to the number of multiple groups of transducer elements L (for example, L₁ to L₉₆), with one ends thereof connected to the main body of the apparatus. The one ends to the first to the n^{th} signal sub-lines sl (sl₁ to slₙ) can be connected to the other ends of the signal lines SL, and the other ends thereof are respectively connected to the first to the n^{th} subgroups of transducer elements T (T₁ to Tₙ). Furthermore, the selection means (connector C) has a number of first switching means (switches c) equivalent to the number of the first to the n^{th} signal sub-lines sl (sl₁ to slₙ). The first switching means (switches c) switch the connection and disconnection of the first to the n^{th} n signal sub-lines sl (sl₁ to slₙ) with the signal line SL. The selection means (connector C) then selects a subgroup of transducer elements T (T₁ to Tₙ) to be activated based on an external activation signal, by switching the respective first switching means (switches c).

Furthermore, the ultrasound probe 1 in the ultrasound diagnosis apparatus 100 in the embodiment has signal lines SL and the first to the n^{th} signal sub-lines sl. The number of signal lines is equivalent to the number of multiple groups of transducer elements L (for example, L₁ to L₉₆), with one ends thereof connected to the main body of the apparatus. One ends of the first to the n^{th} signal sub-lines sl (sl₁ to slₙ) can be connected to the other end of the signal line SL, and the other ends thereof are respectively connected to the subgroups of transducer elements T (T₁ to Tₙ). Furthermore, the selection means (connector C) has a number of first switching means (switches c) equivalent to the number of the first to the nth signal sub-lines sl (sl₁ to slₙ). The first switching means (switches c) switch the connection and disconnection of the first to the nth signal sub-lines sl (sl₁ to slₙ) with the signal line SL. Furthermore, the selection control means (switch controller 91) selects a subgroup of transducer elements T (T₁ to Tₙ) to be activated based on an activation signal from the transmission/reception means (transmitter/receiver 3), by switching the respective first switching means (switches c).

Configuring the equipment in this manner allows only the transducer elements that are positioned in the direction in which the operator wishes to transmit/receive ultrasound for each of the multiple groups of transducer elements L to be activated, resulting in that it is difficult to form a grating lobe. As a result, it is possible to reduce the frequency with which false images occur in an ultrasound diagnostic image. In addition, because the first switching means and the first to the nth signal sub-lines sl are provided, it is sufficient to have only one signal line SL per group of transducer elements L. As a result, since there is no change in the number of signal lines SL even for cases in which the number of transducer elements t is increased in order to achieve a high spatial resolution, the cable do not need to be any thicker.

### SECOND EMBODIMENT

Next, the following is a description of the ultrasound diagnosis apparatus in the second embodiment, with reference to FIGS. 9 and 10.

FIG. 9 is a block diagram illustrating an outline of the ultrasound diagnosis apparatus in order to describe the embodiment. FIG. 9 illustrates only a single group of transducer elements L. It also omits part of the structure. FIG. 10 is a flow chart indicating the operation of the ultrasound diagnosis apparatus. Aspects of the configuration that are the same as the first embodiment are omitted from the description.

### [Configuration of Ultrasound Probe]

In the embodiment, the ultrasound probe 1 comprises: multiple groups of transducer elements L (for example, 96 groups); the number of signal lines SL′ equivalent to the number of the multiple groups of transducer elements L; signal sub-lines sl′ corresponding to subgroups of transducer elements T included in each group of transducer elements L; the number of connectors C′ equivalent to the number of the multiple groups of transducer elements L; and a switch controller 91. FIG. 9 illustrates one group of transducer elements L, one signal line SL′, one connector C′ and signal sub-lines sl′₁ to sl′₃. Furthermore, the transducer elements t used in the embodiment are MUT elements.

One end of the signal line SL′ is connected to the transmitter/receiver 3 (transmitter 31 and receiver 32) in the main unit 2, and the other end thereof is connected to the signal sub-lines to sl′₃. The signal line SL′ is connected via inside of a cable (not illustrated) to the transmitter/receiver 3.

One ends of the signal sub-lines sl′₁ to sl′₃ are connected to the other end of the signal line SL′, and the other ends thereof are respectively connected to the subgroups of transducer elements T₁ to T₃. In general, the signal sub-lines sl′ include the first signal sub-line sl′₁ to the n^{th} signal sub-line sl′ₙ (where n≧2) according to the number of subgroups of transducer elements T.

The connector C′ is used to selectively activate a specified subgroup of transducer elements from among the multiple subgroups of transducer elements T, based on an external activation signal from the main unit 2, etc. Specifically, the connector C′ is configured to include a number of switches c′ equivalent to the number of signal sub-lines sl′. In the embodiment, three switches c′₁ to c′₃ are provided corresponding to the signal sub-lines sl′₁ to sl′₃, respectively. The switches c′ are designed to selectively apply a bias voltage from the bias supply 15 (see below) to the first to the n^{th} subgroups of transducer elements. It is sufficient if at least one connector C′ is provided with regard to the multiple groups of transducer elements L. The connector C′ in the embodiment is one example of the “selection means.” Furthermore, the switches c′₁ to c′₃ in the embodiment are examples of the “second switching means.”

The switch controller 91 controls the operation of the connector C′. Specifically, the switch controller 91 performs the switching of each of the switches c′. For example, when a delay signal is transmitted from the controller 9 in order to activate the subgroup of transducer elements T₁ alone, the switch controller 91 activates the switch c′₁ based on the delay information, connecting the bias supply 15 (see below) and the signal sub-line sl′₁. In this state, the bias supply 15a applies a bias voltage via the signal sub-line sl′₁ to the subgroup of transducer elements T₁. As a result, only the subgroup of transducer elements T₁ receives the signal (the activation signal) transmitted from the transmitter 31 (first pulsar 31a), and is able to transmit ultrasonic waves. There is no restriction requiring the switch controller 91 to only operate one switch c′. One switch controller 91 may simultaneously operate multiple switches c′ (for example, c′₁ and c′₂). As a result, it is possible for multiple subgroups of transducer elements (for example T₁ and T₂) to be simultaneously activated. The switch controller 91 in the embodiment is an example of the “selection control means.”

It is sufficient if at least one switch controller 91 is provided with regard to the multiple groups of transducer elements L. In this case, the subgroup of transducer elements T (for example T₁) belonging to each of the groups of transducer elements L (for example L₁ to L₉₆) is commonly connected. Additionally, if the switch controller 91 is provided for each of the multiple groups of transducer elements L, differing controls can be performed for each group of transducer elements L. For example, it is possible to operate the switch c′₁ in order to activate only the subgroup of transducer elements T₁ within the group of transducer elements L₁, and to operate the switch c′₃ in order to activate only the subgroup of transducer elements T₃ within the group of transducer elements L₂.

### <Configuration of Main Unit>

In the embodiment, the main unit 2 comprises a bias supply 15. The bias supply 15 generates a bias voltage to be applied to the subgroups of transducer elements T (transducer elements t) via the controller C′, based on the delay signal from the controller 9.

### <Operation>

Next, the following is a description of the operation of the ultrasound diagnosis apparatus 100 in the embodiment, with reference to FIG. 10. Here, the reference direction P is predetermined.

The controller 9 determines the ultrasound transmission direction S based on the scan mode of ultrasound beams (S20).

The calculator 92 calculates the extent (angle θ) to which the ultrasound transmission direction S determined in S20 is displaced from the reference direction P (S21).

The controller 9 determines the transducer elements t to be activated based on the calculation results in S21, from table data, etc. (S22). Here, it is assumed that the transducer elements t₁ are selected.

The controller 9 transmits a delay signal and delay information based on the determination result by 22 to the bias supply 15, the switch controller 91 and the transmitter/receiver 3. The switch controller 91 activates the switch c′₁ based on the delay information, connecting the bias supply 15 and the signal sub-line sl′₁ (S23). As a result, a bias voltage is applied to the transducer elements t₁ via the signal sub-line sl′₁, making it possible to operate the transducer elements t₁ (S24).

The transmitter 31 of the transmitter/receiver 3 activates the first pulsar 31a based on the delay signal, and transmits the activation signal to the transducer elements t₁. Based on the activation signal, the transducer elements t₁ transmit ultrasonic waves to the subject (S25).

The receiver 32 of the transmitter/receiver 3 activates the first gain part 32a based on the delay signal, then applies the specified gain to the echo signal and receives it (S26).

The echo signal to which gain has been applied in S15 is transmitted to the signal processor 4, etc., (not illustrated in FIG. 9), and once it is subjected to the specified processing (S27), the composition part 6 acquires image data (S28). The display 81 displays an image based on the image data acquired in S28.

### <Operation and Effect>

The following is an explanation of the operation and effect of the embodiment.

The transducer elements included in the ultrasound probe 1 in the embodiment are MUT elements. The selection means (connector C′) has a number of second switching means (switches c′) equivalent to the number of the first to the n^{th} subgroups of transducer elements T (T₁ to Tₙ). The second switching means (switches c′) selectively apply a bias voltage from the bias supply 15 to the first to the n^{th} subgroups of transducer elements T (T₁ to Tₙ). Then, the selection means (connector C′) selects, by switching of the respective second switching means (switch c′), a subgroup of transducer elements T (T₁ to Tₙ) to be activated based on an external activation signal.

Furthermore, the transducer elements t included in ultrasound diagnosis apparatus 100 in the embodiment are MUT elements. The selection means (connector C′) has a number of second switching means (switches c′) equivalent to the number of the first to the n^{th} subgroups of transducer elements T (T₁ to Tₙ). The second switching means (switches c′) selectively apply the bias voltage from the bias supply 15 to the first to the nth subgroups of transducer elements T (T₁ to Tₙ). The selection control means (switch controller 91) selects, by switching of the respective second switching means (switches c′), the subgroup of transducer elements T (T₁ to Tₙ) to be activated based on the activation signal from the transmission/reception means (transmitter/receiver 3).

By selecting a bias voltage to be applied to the transducer elements in this way, only the transducer elements t in the direction in which the operator wishes to transmit/receive ultrasound are activated for each of the multiple groups of transducer elements L, thereby it is difficult to form a grating lobe. As a result, it is possible to reduce the frequency with which false images occur in an ultrasound diagnostic image. In addition, because the second switching means and the first to the n^{th} signal sub-lines sl′ are provided, it is sufficient to only have one signal line SL′ per group of transducer elements L. As a result, since there is no change in the number of the signal lines SL″ even for cases in which the number of the transducer elements t is increased, the cables do not need to be any thicker.

### THIRD EMBODIMENT

Next, the following is a description of the ultrasound diagnosis apparatus in the third embodiment, with reference to FIGS. 11 and 12. FIG. 11 is a block diagram illustrating an outline of the ultrasound diagnosis apparatus in order to describe the embodiment. FIG. 11 illustrates only a single group of transducer elements L. It also omits part of the structure. FIG. 12 is a flow chart indicating the operation of the ultrasound diagnosis apparatus. Aspects of the configuration that are the same as the first and second embodiments are omitted from the description.

### <Configuration of Ultrasound Probe>

In the embodiment, the ultrasound probe 1 comprises multiple groups of transducer elements L (for example, 96 groups), along with the number of signal lines SL″ equivalent to the number of subgroups of transducer elements T included in each group of transducer elements L. FIG. 11 illustrates one group of transducer elements L (subgroups of transducer elements T₁ to T₃), and three signal lines SL″₁ to SL″₃.

One ends of the signal lines SL″ are connected to the subgroups of transducer elements T, while the other ends thereof can be connected via the connector C″ (see below) provided in the main unit 2 to the transmitter/receiver 3 (transmitter 31, receiver 32)

### <Configuration Of Main Unit>

In the embodiment, the main unit 2 comprises a connector C″. The connector C″ is connected to the transmitter 31, for the case in which a specified subgroup of transducer elements is selectively activated from among the multiple subgroups of transducer elements T based on an activation signal from the transmitter 31. Furthermore, the connector C″ is connected to the receiver 32, for the case in which the echo signal acquired by the subgroups of transducer elements T (transducer elements t) are received. Specifically, the connector C″ comprises a number of switches c″, equivalent to the number of the signal lines SL″. In this embodiment, three switches c″₁ to c″₃ are provided corresponding to the signal lines SL″₁ to SL″₃, respectively. The switches c″ switch the connection of the signal lines SL″ with the transmitter 31 and receiver 32. The connector C″ in this embodiment is an example of the “selection means.”

In the embodiment, the controller 9 includes a timing controller 93.

The timing controller 93 controls the timing of the switching of the connection of the signal lines SL″ with the transmitter 31 and the receiver 32. For example, the timing controller 93 sends delay information to the switch controller 91 in order to connect the signal line SL″₁ and the transmitter 31 (first pulsar 31a) based on the delay signal. Furthermore, when it detects that an activation signal is sent from the transmitter 31, the timing controller 93 transmits delay information to the switch controller 91 in order to connect the signal line SL″₁ and the receiver 32 (first gain part 32a). The switch controller 91 controls the operation of the connector C″ (switches c″) based on the delay information from the timing controller 93.

### <Operation>

Next, the following is a description of the operation of the ultrasound diagnosis apparatus 100 in the embodiment, with reference to FIG. 12. Here, the reference direction P is predetermined.

The controller 9 determines the ultrasound transmission direction S based on the scan mode of ultrasound beams (S30).

The calculator 92 calculates the extent (angle θ) to which the ultrasound transmission direction S defined in S30 is displaced from the reference direction P (S31).

The controller 9 determines the transducer elements t to be activated based on the calculation results in S31, from table data, etc. (S32). Here, it is assumed that the transducer elements t₁ are selected.

The controller 9 transmits a delay signal based on the determination result by S32 to the timing controller 93, the transmitter 31 and the receiver 32. The timing controller 93 transmits delay information to the switch controller 91 in order to connect the signal line SL″₁, to which the transducer elements t₁ are connected based on the delay signal, and the first pulsar 31a inside the transmitter 31. The switch controller 91 activates the switches c″₁ based on the delay information, connecting the signal line SL″₁ and the first pulsar 31a (S33).

The transmitter 31 activates the first pulsar 31a based on the delay signal, and transmits an activation signal to the transducer elements t₁. Based on the activation signal, the transducer elements t₁ transmit ultrasonic waves to the subject (S34).

Then, upon detecting that an activation signal is sent from the first pulsar 31a, the timing controller 93 transmits a connection signal to the switch controller 91 in order to connect the signal line SL″₁ and the first gain part 32a. The switch controller 91 switches the switches c″₁ based on the connection signal, causing the signal line SL″₁ and the first gain part 32a to be connected (S35).

The receiver 32 of the transmitter/receiver 3 activates the first gain part 32a based on the delay signal, then applies the specified gain to the echo signal and receives it (S36).

The echo signal to which gain has been applied in S36 is transmitted to the signal processor 4, etc., (not illustrated in FIG. 11), and once it has been subjected to the specified processing (S37), the composition part 6 acquires image data (S38). The display 81 displays an image based on the image data acquired in S38.

### <Operation and Effect>

The following is an explanation of the operation and effect of the embodiment.

The ultrasound diagnosis apparatus 100 in the embodiment comprises an ultrasound probe 1, a transmission means (the transmitter 31), a receiving means (the receiver 32), a selection means (the connector C″), a timing control means (the timing controller 93) and a selection control means (the switch controller 91). The ultrasound probe 1 has a transducer element unit in which multiple groups of transducer elements L (for example, L₁ to L₉₆) are arranged. Each of the multiple groups of transducer elements (for example, L₁ to L₉₆) has the first to the n^{th} subgroups of transducer elements T (T₁ to Tₙ), and the emitting surfaces of the transducer elements t included in the first to the n^{th} subgroups of transducer elements T (T₁ to Tₙ) are arranged such that they faces different directions for each subgroups of transducer elements T. The transmission means (transmitter 31) causes the transmission of ultrasonic waves by transmitting an activation signal to the transducer elements t included in the first to the n^{th} subgroups of transducer elements T. The receiving means (receiver 32) receives an echo signal based on the transmitted ultrasonic waves. In the event that the specified subgroup of transducer elements T from among the first to the n^{th} subgroups of transducer elements T (T₁ to Tₙ) is selectively activated based on the activation signal, the selection means (connector C″) is connected to the transmission means (transmitter 31), while in the event that the echo signal is received, it is connected to the receiver means (receiver 32). The timing control means (timing controller 93) controls the timing of switching the connection of the selection means (connector C″) with the transmission means (transmitter 31) and the receiving means (receiver 32). The selection control means (switch controller 91) controls the operation of the selection means (connector C″) based on a signal from the timing control means (timing controller 93).

By configuring in this manner, only the transducer elements in the direction in which the operator wishes to transmit/receive ultrasound are activated for each of the multiple groups of transducer elements L, thereby it is difficult to form a grating lobe. As a result, it is possible to reduce the frequency with which false images occur in an ultrasound diagnostic image.

### Examples

The following is a description of specific examples and comparative examples of the embodiments described above, with reference to FIGS. 13A through 16B.

### Comparative Example

The comparative example has a configuration in which a transducer element α is arranged on a base 13. FIG. 13A is a diagram illustrating the directivity of ultrasonic waves transmitted from the transducer element α. FIG. 13A illustrates the directivity when the transducer element α is viewed from the X-Z direction.

FIG. 13B is a graph illustrating the relationship between the angle at which ultrasonic waves are transmitted and the pressure of ultrasonic waves (acoustic pressure) in the configuration illustrated in FIG. 13A. The vertical axis of the graph represents the pressure of ultrasonic waves (acoustic pressure). The horizontal axis of the graph represents the angle at which the ultrasonic waves are transmitted. Here, the center of the horizontal axis (the Z direction) is defined as an angle of 0°, the +X direction is defined as a positive angle, and the −X direction is defined as a negative angle.

As illustrated in FIG. 13A, it is necessary to ensure a broad level of directivity of the transducer element a in order to generate ultrasonic waves across a broad area using a single transducer element. For this reason, as illustrated in FIG. 13B, even if the ultrasound transmission angle increases (or decreases), ultrasonic waves will be transmitted at a high pressure (acoustic pressure) to a certain extent.

In this case, however, a grating lobe may be formed in a different direction from the direction in which the operator wishes to generate ultrasonic waves. This grating lobe may be the cause of the formation of false images on the ultrasound diagnostic image.

### Example

FIGS. 14A, 15A and 16A illustrate the directivity of ultrasonic waves transmitted from the transducer elements t (t₁ to t₃) provided on the base 12. Here, the directivity is illustrated when the transducer elements t are viewed from the X-Z direction. FIGS. 14A, 15A and 16A illustrate only the transducer elements t to be activated.

FIGS. 14B, 15B and 16B are graphs illustrating the relationship between the angle at which the ultrasonic waves are transmitted and the pressure of the ultrasonic waves (acoustic pressure) in the configuration illustrated in FIGS. 14A, 15A and 16A. The vertical axes of the graphs represent the pressure of the ultrasonic waves (acoustic pressure). The horizontal axes of the graphs represent the angle at which the ultrasonic waves are transmitted. Here, the center of the horizontal axis (the Z direction) is defined as an angle of 0°, the +X direction is defined as a positive angle, and the −X direction is defined as a negative angle.

As illustrated in FIGS. 14A and 14B, when the transducer elements t₂ are activated, the pressure (acoustic pressure) of the ultrasonic waves transmitted in the direction in which the emitting surfaces of transducer elements t₂ face (direction of angle 0°) increases. As the sizes of the transducer elements t₂ are smaller than those in the comparative example, the directivity of the acoustic pressure created by the transducer elements t₂ becomes broader. However, the beam direction formed by activating the transducer elements t₂ is substantially the vicinity of the Z direction (reference direction P), making it difficult for a grating lobe to be formed.

As can be seen from FIG. 15A, even if the transducer elements t₁ and the transducer elements t₂ are activated, the pressure (acoustic pressure) of ultrasonic waves transmitted in the facing directions of the emitting surfaces of the transducer elements t₁ and the transducer elements t₂ becomes higher than other directions (see FIG. 15B). It can be similarly seen that in cases such as that in FIG. 16A, when only the transducer elements t₁ are activated, the pressure (acoustic pressure) of ultrasonic waves transmitted in the facing direction of the emitting surfaces of the transducer elements t₁ becomes higher than other directions (see FIG. 16B). For this reason, it is difficult for a grating lobe to be formed in a direction other than that in which the operator wishes to generate ultrasonic waves.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

### EXPLANATION OF SYMBOLS

1 Ultrasound probe
2 Main unit
3 Transmitter/receiver
4 Signal processor
5 Image generator
6 Composition part
7 Display controller
8 User interface (UI)
9 Controller
10 Transducer element unit
11 Seat
12 Base
13 Surface
14 Groove
31 Transmitter
31a First pulsar
31b Second pulsar
31c Third pulsar
32 Receiver
32a First gain part
32b Second gain part
32c Third gain part
81 Display
82 Operation part
91 Switch controller
92 Calculator
100 Ultrasound diagnosis apparatus
C Connector
L Group of transducer elements
T Subgroup of transducer elements
c Switch
t Transducer element

What is claimed is:

1. An ultrasound probe, comprising a transducer element unit in which multiple groups of transducer elements are arranged, wherein
each of the multiple groups of transducer elements comprises a first to n^{th} subgroups of transducer elements,
emitting surfaces of the transducer elements included in the first to the n^{th} subgroups of transducer elements are arranged such that they face different directions for each subgroup of transducer elements; and comprising
a selection means that selectively activates a specific subgroup of transducer elements from among the first to the n^{th} subgroups of transducer elements, based on an external activation signal.

2. The ultrasound probe according to claim 1, comprising:
multiple signal lines, the number of which is equivalent to the number of the multiple groups of transducer elements, and one ends of which are connected to the main body of the apparatus; and
a first to n^{th} signal sub-lines, one ends of which can be connected to the other ends of the signal lines, while the other ends are respectively connected to the first to the n^{th} subgroups of transducer elements, wherein
the selection means comprises first switching means, the number of which is equivalent to the number of the first to the n^{th} signal sub-lines, that switch connection and disconnection of the first to the n^{th} signal sub-lines with the signal lines, and
the selection means selects, by switching the respective first switching means, the subgroup of transducer elements to be activated based on the external activation signal.

3. The ultrasound probe according to claim 1, wherein
the transducer elements are MUT elements,
the selection means comprises second switching means, the number of which is equivalent to the number of the first to the n^{th} signal sub-lines, that selectively apply a bias voltage from a bias supply to the first to the n^{th} subgroups of transducer elements, and
the selection means select, by switching the respective second switching means, the subgroup of transducer elements to be activated based on the external activation signal.

4. The ultrasound probe according to claim 1, wherein each of the multiple groups of transducer elements comprises either a convex or a concave base on which the first to the n^{th} subgroups of transducer elements are arranged.

5. The ultrasound probe according to claim 4, wherein the bases are arranged either in a row or a matrix on the transducer element unit.

6. The ultrasound probe according to claim 4, wherein grooves are formed between the multiple bases in the transducer element unit.

7. The ultrasound probe according to claim 5, wherein grooves are formed between the multiple bases in the transducer element unit.

8. The ultrasound probe according to claim 2, wherein each of the multiple groups of transducer elements comprises either a convex or a concave base on which the first to the n^{th} subgroups of transducer elements are arranged.

9. The ultrasound probe according to claim 8, wherein the bases are arranged either in a row or a matrix on the transducer element unit.

10. The ultrasound probe according to claim 8, wherein grooves are formed between the multiple bases in the transducer element unit.

11. The ultrasound probe according to claim 9, wherein grooves are formed between the multiple bases in the transducer element unit.

12. The ultrasound probe according to claim 3, wherein each of the multiple groups of transducer elements comprises either a convex or a concave base on which the first to the n^{th} subgroups of transducer elements are arranged.

13. The ultrasound probe according to claim 12, wherein the bases are arranged either in a row or a matrix on the transducer element unit.

14. The ultrasound probe according to claim 12, wherein grooves are formed between the multiple bases in the transducer element unit.

15. The ultrasound probe according to claim 13, wherein grooves are formed between the multiple bases in the transducer element unit.

16. An ultrasound diagnosis apparatus, comprising:
an ultrasound probe that comprises a transducer element unit in which multiple groups of transducer elements are arranged, wherein each of the multiple groups of transducer elements comprises a first to n^{th} subgroups of transducer elements, and emitting surfaces of the transducer elements included in the first to the n^{th} subgroups of transducer elements are arranged such that they face different directions for each subgroup of transducer elements, and comprises a selection means that selectively activates a specific subgroup of transducer elements from among the first to the n^{th} subgroups of transducer elements, based on an external activation signal;
a transmission/reception means that carries out transmission/reception of ultrasonic waves by sending the activation signal to transducer elements included in the first to the n^{th} subgroups of transducer elements; and
a selection control means that controls the operation of the selection means.

17. The ultrasound diagnosis apparatus according to claim 16, wherein
the ultrasound probe comprises:
multiple signal lines, the number of which is equivalent to the number of the multiple groups of transducer elements, and one ends of which are connected to the main body of the apparatus; and
a first to n^{th} signal sub-lines, one ends of which can be connected to the other ends of the signal lines, while the other ends are respectively connected to the first to the n^{th} subgroups of transducer elements,
the selection means comprises first switching means, the number of which is equivalent to the number of the first to the n^{th} signal sub-lines, that switch connection and disconnection of the first to the n^{th} signal sub-lines with the signal lines, and
the selection control means selects, by switching the respective first switching means, the subgroup of transducer elements to be activated based on the activation signal from the transmission/reception means.

18. The ultrasound diagnosis apparatus according to claim 16, wherein
the transducer elements are MUT elements,
the selection means comprises second switching means, the number of which is equivalent to the number of the first to the n^{th} signal sub-lines, that selectively apply a bias voltage from a bias supply to the first to the n^{th} subgroups of transducer elements, and
the selection control means selects, by switching the respective second switching means, the subgroup of transducer elements to be activated based on the activation signal from the transmission/reception means.

19. An ultrasound diagnosis apparatus, comprising:
an ultrasound probe that comprises a transducer element unit in which multiple groups of transducer elements are arranged, wherein each of the multiple groups of transducer elements comprises a first to n^{th} subgroups of transducer elements, and emitting surfaces of the transducer elements included in the first to the n^{th} subgroups of transducer elements are arranged such that they face different directions for each subgroup of transducer elements;
a transmission means that causes ultrasonic waves to be transmitted by sending an activation signal to the transducer elements included in the first to the n^{th} subgroups of transducer elements;
a reception means that receives echo signals based on the transmitted ultrasonic waves;
a selection means that is connected to the transmission means in the case that a specific subgroup of transducer elements from among the first to the n^{th} subgroups of transducer elements is selectively activated based on the activation signal, and connected to the reception means in the case that the echo signals are received;
a timing control means that controls the timing of switching between the connection with the transmission means and the connection with the reception means in the selection means; and
a selection control means that controls the operation of the selection means based on a signal from the timing control means.
